(19) United States
(12) Patent Application Publication
(10) Pub. No.: US 2026/0210935 A1
(43) Pub. Date: Jul. 23, 2026

(54) METHODS AND SYSTEM FOR MONITORING WELL CONDITIONS

(71) Applicant: RESMAN AS, Ranheim (NO)

(72) Inventors: Olaf Kristoffer HUSEBY, Ranheim (NO); Gaute OFTEDAL, Ranheim (NO)

(21) Appl. No.: 19/140,051

(22) PCT Filed: Dec. 19, 2023
(86) PCT No.: PCT/EP2023/086501

§ 371 Date: Jun. 17, 2025

(30) Foreign Application Priority Data
Dec. 19, 2022 (GB) 2219174.6

(51) Int. Cl. G01N 33/28; E21B 47/10; E21B 49/08

(52) U.S. Cl. CPC G01N 33/2823; E21B 47/10; E21B 49/08

(57) ABSTRACT

The invention provides a system and methods of monitoring at least one characteristic and/or condition related to a hydrocarbon well, wherein a well comprises at least one tracer source located at a known zone or section in a well. The method comprises obtaining measured tracer data of tracer in the well fluid wherein the at least one tracer source is configured to release tracer material to the well fluid as a function of the at least one characteristic and/or condition of at least one zone or section of the well. The method comprises obtaining or calculating expected tracer data; and comparing the measured tracer data with the expected tracer data to determine at least one characteristic and/or condition of at least one zone or section of the well.

The present invention relates to systems and methods for well monitoring. Aspects of the invention include methods and systems to facilitate measurement and monitoring of conditions and/or characteristics of a well system.

### BACKGROUND TO THE INVENTION

The efficient recovery of hydrocarbons from a reservoir is a difficult and complex process which requires an understanding of the flow conditions of the hydrocarbons in the reservoir and the well systems connected to the reservoir.

Downhole tracers released into the production flow in a producing well have been previously used for estimating which fluids flow in parts of the well. Methods of monitoring fluid rate based on transient flow where distinct tracers are arranged at different influx zones in a well are known.

EP2633152 discloses a method of estimating influx profile for well fluids to petroleum well. The method comprises locating tracers into a well and inducing a transient in the production rate of the entire production flow by shutting in the well. The well is shut-in for a period of time to allow a high concentration of tracers to build up in the well and then the well is re-started to carry the tracers to surface. Sampling and analysis of the concentration of the different tracers is used to provide qualitative and quantitative production data.

### SUMMARY OF THE INVENTION

It is amongst the aims and objects of the invention to provide a method and system for monitoring or measuring at least one characteristic and/or condition of a hydrocarbon well.

It is another object of the present invention to provide a method and system for estimating the release of tracer over time into a well to allow characteristics and/or conditions of at least one zone or section of a well system to be monitored.

It is a further object of an aspect of the invention to a provide a system and/or method which is capable of detecting and/or estimating changes in well-related conditions.

It is another object of the present invention to a provide a system and/or method which is capable of detecting and/or estimating changes in a well system and/or reservoir connected to the well system.

Further aims and objects of the invention will become apparent from reading the following description.

According to a first aspect of the invention there is provided a method of monitoring at least one characteristic and/or condition related to a hydrocarbon well, wherein the well comprises at least one tracer source located at a known level of the well, the method comprising;
obtaining measured tracer data of tracer in a well fluid; wherein the at least one tracer source is configured to release tracer material to the well fluid as a function of the at least one characteristic and/or condition of at least one zone or section of the well.

Each of the at least one tracer source may have unique tracer material for each tracer location. Each of at least one tracer sources may be located at different known levels of the well. The method may comprise measuring for tracer released from the at least one tracer source. The method may comprise obtaining or calculating expected tracer data of the at least one tracer source. The expected tracer data for at least one tracer source may be calibrated to or using the measured tracer data. The expected tracer data for at least one tracer source may be calibrated to or using measured tracer signal/data points. The expected tracer data for at least one tracer source may be based on at least one physical parameter of the well.

The expected tracer data for the at least one tracer source may be based on experiments and/or field data. The expected tracer data for at least one tracer source may be based on tracer data from the well and/or a section or zone with the tracer source(s). The expected tracer data for at least one tracer source may be based on another well or more than one well and/or the reservoir. The expected tracer data for at least one tracer source may be based on modelled data. The expected tracer data for at least one tracer source may be based on computer learning, computer program and/or algorithms.

The method may comprise comparing a measured tracer data set or data point with an expected tracer data set or data point to determine at least one characteristic and/or condition of the well. The method may comprise comparing measured tracer data with the expected tracer data to determine at least one characteristic and/or condition of the well and/or to determine at least one characteristic and/or condition of at least one zone or section of the well. The method may comprise observing one or more deviations between the measured tracer data set or data point and the expected tracer data set or data point. The method may comprise comparing a measured tracer data with an expected tracer data to determine at least one characteristic and/or condition of the well at the location, or in the vicinity of the at least one tracer source. The expected tracer data may be an expected tracer response as a function of a characteristic or condition of the well or reservoir. By comparing the expected tracer data with the measured tracer data a characteristic or condition of the well or reservoir may be determined. The method may comprise measuring at least one parameter of the well and/or at least one parameter of a zone or section of the well where the at least one tracer source is located. The method may comprise comparing the at least one parameter data with the measured tracer data and/or expected tracer data. The method may comprise associating changes in the well parameter data with changes in the measured tracer data. The method may comprise associating changes in the well parameter data with one or more deviations between the measured tracer data and the expected tracer data.

The method may comprise inferring one or more deviations between the measured tracer data and the expected tracer data corresponds with changes in at least one characteristic and/or condition of at least one zone or section of the well. The method may comprise inferring at least one characteristic and/or condition of at least one zone or section of the well from one or more deviations between the measured tracer data and the expected tracer data.

The at least one well parameter, or well-related parameter may be selected from the group comprising pressure, temperature, ion data, flowrate, flowrate of a target and/or non-target fluid, viscosity, mass-density, solid components in the fluid, gas-rate, choke settings, water cut, gas to oil ratio; composition of a target or non-target fluid, well fluid type, injection rate, production rate, production rate of a nearby producing well, seismic activity in the vicinity of the well and/or reservoir, electromagnetic signal data, tracer data and/or core sample data.

The method may comprise measuring at least one tracer data point. The at least one tracer data point may comprise tracer concentration, tracer mass, mass flow and/or tracer type. The method may comprise measuring at least one tracer concentration of at least one tracer in the well fluid. The measured tracer data may be selected from the group comprising tracer concentration, amount, tracer mass, mass flow and/or tracer type, tracer function, release function and/or rate of tracer released as a function of the at least one characteristic and/or condition of the well. The expected tracer data may be selected from the group comprising expected tracer concentration, amount, mass, mass flow, tracer type, expected tracer function, expected release function and/or expected rate of tracer released as a function of the at least one characteristic and/or condition of the well.

The method may comprise obtaining tracer data by measuring for the at least one tracer in the well fluid. The method may comprise obtaining tracer data by measuring for the presence and/or concentration of the at least one tracer in the well fluid. The method may comprise obtaining tracer data from sampling well fluid. The method may comprise obtaining tracer data from samples previously collected from the well. The method may comprise obtaining tracer data by prediction or provided from data files, documents, or physical documents or text or figures. For example, a data point could have been defined, obtained or procured by a third party, and then later applied in this invention.

The release of tracer from the at least one tracer source may be dynamic. The tracer release functionality may be dynamic. The release rate to a well fluid may be as a function of the at least one characteristic and/or condition of the well. The at least one characteristic and/or conditions may be selected from the group comprising pressure, temperature, viscosity of a well fluid, a composition of a target or a non-target fluid, diffusion coefficient of the tracer, diffusion coefficient (D_m) of the tracer in a target fluid, tracer type; composition of a tracer matrix; composition of a tracer material; physical dimensions of the tracer system, molecular weight of tracer, water cut, well fluid type, and/or degree of exposure of tracer to target fluid.

The at least one characteristic and/or condition may be a downhole well characteristic and/or condition. The at least one characteristic and/or condition may be a well fluid characteristic and/or condition. The at least one characteristic and/or condition may be a reservoir characteristic and/or condition. The at least one characteristic and/or condition may be a related well characteristic and/or condition. The release rate of the tracer material to a well fluid may be dependent on two or more characteristics and/or conditions of the well.

The well may comprise one or more well in a well system. The well may be production well. The production well may comprise at least one tracer source located at a known level in the production well. The well may be an injection or injector well. The injection or injector well may comprise at least one tracer source located at a known level in the injection or injector well. The well may be a producer well. The producer well may comprise at least one tracer source located at a known level in the producer well. The injection and/or producer well may comprise at least one tracer source located at a known level in the injection and/or producer well. Each tracer source may be located in a known level in the well.

The method may comprise modelling an operation of the well with a physics based model of the well system to obtain modelled tracer release function for the at least one characteristic and/or condition of the well. The method may comprise generating a library or database of tracer release function profiles for each well characteristic and/or condition. The method may comprise comparing at least one measured tracer concentration with the library or database of tracer release function profiles to identify the at least one characteristic and/or condition of the well.

The tracer source may be injected into the well. The tracer source may be a part of the run-in-hole completion. The tracer source may be stationed or positioned outside of the downhole part of the well itself for example in the template or wellhead or part of a coiled tubing or production tool.

Each tracer source may comprise at least one tracer material. The tracer material may be selected from the group comprising chemical, fluorescent, phosphorescent, magnetic, DNA and radioactive compounds. The tracer material may comprise chemical tracers selected from the group comprising perfluorinate hydrocarbons or perfluoroethers. The perfluorinated hydrocarbons may be selected from the group of perfluoro butane (PB), perfluoro methyl cyclopentane (PMCP), perfluoro methyl cyclohexane (PMCH). The tracer material may be a solid, liquid and/or a gas form. The tracer material may be a powder or dissolved or mixed with other components. The tracer material may be in a concentrated form. The tracer source may comprise a tracer and a carrier. The carrier may be a matrix material. The carrier may be dissolvable or erodable substance. The carrier may comprise polymer, proppant, metal, and/or salt. The carrier may be a polymeric material. The tracer material may be inside, partially encapsulated and/or encapsulated by the carrier. The tracer material may be uniformly distributed in the carrier. The tracer material may be inhomogeneously distributed in the carrier. The tracer material may be chemically immobilized within and/or to the carrier. The tracer material may be chemically immobilized by a chemical interaction between the tracer and the carrier. The tracer material may be chemically immobilized in a way that it releases tracer molecules or particles in the presence of a chemical and/or physical trigger. The tracer material may be chemically immobilized in a way that it releases tracer molecules or particles in the presence of a specific characteristic and/or condition in the well. By varying the chemical and/or physical interactions between the tracer and the polymer the release mechanism and the rate of release of tracer molecules from the tracer material may be controlled. The tracer material may be released from the tracer carrier with a dynamic release rate. The tracer material may be released from the tracer carrier with an even release rate. The tracer material may be released from the tracer carrier at a release rate dependent on at least one characteristic and/or condition of the well. The carrier may be a selected from poly methyl methacrylates (PMMA), poly methylcrylates, poly ethylenglycols (PEG), poly lactic acid (PLA) or poly glycolic acid (PGA) commercially available polymers or copolymers thereof. The carrier may be selected from polymers with higher rates of tracer molecules release such as polyethylene and polypropylene. The tracer may be physically dispersed and/or physically encapsulated in the carrier. The tracer material may release tracer molecules into fluid by dissolution or degradation of the carrier and/or the tracer into the fluid. The carrier may be selected to controllable degrade on contact with a fluid. The carrier may be selected to controllable degrade when exposed to a change in a physical condition and/or characteristic of a well. The carrier may be selected to degrade by hydrolysis of the carrier. The tracer material and/or the carrier may be fluid specific such that the tracer molecules will be released from the tracer material at a different rate in a response to a contact with a target liquid. The tracer material and/or the carrier may be fluid specific such that the tracer molecules will be released from the tracer material in a response to a contact with a target liquid. The tracer material and/or the carrier may be well condition and/or condition specific such that the tracer molecules will be released at different rate from the tracer source in response to a change of physical condition or characteristic in a well in which the tracer source is located. The tracer material and/or the carrier may be well characteristic specific and/or condition specific such that the tracer molecules will be released at different rate from the tracer source in response to the presence and/or change of a condition or characteristic in a well in which the tracer source is located. The tracer material and/or the carrier may be well characteristic specific and/or condition specific such that the tracer molecules will be released from the tracer source in response to a change of physical condition or characteristic in a section of well in which the tracer source is located. The tracers and/or the carrier may be chemically intelligent such that tracer molecules will be released from the tracer material and/or released from the tracer material at a different rate as a response of specific events, e.g. they may respond to an oil flow (oil-active) but show no response to a water flow (water-resistant). Another group of chemical compounds can be placed in the same region, which release tracers in water flow (water-active) but show no response to an oil flow (oil-resistant). The tracers and/or the carrier may be chemically intelligent such that tracer molecules will be released from the tracer material as a response the exposure of the tracer material to a well fluid and/or a target well fluid. The specific events may be changes in at least one well parameter. The specific events may be present or occurrence of the at least one well parameter. The at least one well parameter may be selected from the group comprising pressure, temperature, ion data, flowrate, flowrate of a target and/or non-target fluid, viscosity, mass-density, solid components in the fluid, gas-rate, choke settings, water cut, gas to oil ratio; a composition of a target or a non-target fluid and/or well fluid type injection rate, injection rate of a well with tracer system/source, injection rate of any related injector or producer hydrocarbon well, production rate of a nearby producing well, seismic activity in the vicinity of the well and/or reservoir, tracer data, electromagnetic signal data and/or core sample data. Tracer can release from the tracer source in an even, frequent, sporadic, declining or increasing, predictable or unpredictable fashion.

The tracer material may be released from the tracer source at a first release rate under a first well condition and/or first well characteristic in the section of the well the tracer source is located. The tracer material may be released from the tracer source at a second release rate under a second well condition and/or second well characteristic in the section of the well the tracer source is located. The first release rate and/or the second release rate may each be even release rates at different release levels i.e the second release rate may be higher or lower than the first release rate but at a consistent rate. The tracer molecules may be detected and its concentration measured by sampling well fluid and/or production fluid. The method may comprise measuring tracer data by sampling the well fluid and/or production fluid. The sampling may be conducted at one or more sampling times. The sampling may be conducted downhole downstream of the tracer release apparatus or at surface. The sampling point where samples are collected may be anywhere downhole, in a completion, in a flowline, topside facility, downstream of topside facilities, for example from the oil stored in an oil tanker, from a refinery, or the flowline between topside facility and to the refinery. Sampling can be for a single sample or for many samples over a short or long time interval, or separated into more than one time interval. Samples may be collected for later analysis. Samples may be collected by devices where tracers are extracted from the fluid. The tracer molecules may be detected by a detection device such a probe. The detection device may facilitate real time monitoring and/or analysis of the tracer in the production fluid. Analysis may be performed at the same location as the sampling such as a temporary or permanently placed onsite lab. Analysis may be performed at a separate location, so that the samples need to be transported there. Analysis can be conducted on data from an online analysis probe in which case it could analyze anywhere a sample could be collected, or as part of a tool on a coiled tubing, or as a part of the permanent completion. Analysis may comprise any tracer type, such as tracers with affinity to one or more phases of oil, water, gas, or of different chemical tracers such as acids, ions, hydrocarbons, organic compounds, radioactive, proteins, DNA, or non-chemical such as a temperature. Analysis may be conducted on a single tracer or one or more tracer types. Analysis may comprise tracer concentrations and/or tracer arrival time. The tracer data analysis may be of units such as concentration in mass per mass (mg/kilo), or mass per volume (mg/cubic meter), volume per volume (cubic meter per liter) or other units, or it can be total mass such as Gram, or as mass-rate such as gram per hour. The analysis device and/or method may be based on analysis principles and/or components such as mass spectrometry, phase separation, tracer extraction, chemical treatment such as a reagent, acid, inhibitor, buffer, or other chemical properties, magnetic or electric scattering, electron, proton, ion, or photon with physical principles such as dispersion, adsorption, advection, convection, or other physical principles. The tracer molecules may be detected and its concentration measured by different techniques such as optical detection, optical fibers, spectrophotometric methods, PCR techniques combined with sequential analysis, chromatographic methods, or radioactivity analysis. The invention is not restricted to the above-mentioned techniques.

Each of the collection, detection, analysis and/or interpretation steps of tracer data in well fluid and/or well parameter data may be considered as separate methods from one another and performed at different times or jurisdictions. The detection, analysis and/or interpretation steps of tracer in well fluid may be separate methods to installation of tracer sources and/or the collection of samples. Samples may be collected and the tracer detected, analysed and/or interpreted at a time or jurisdiction which is separate and distinct from the location of well and therefore the collection of the samples.

According to a second aspect of the invention there is provided a method of monitoring at least one characteristic and/or condition of a hydrocarbon well, the method comprising:
providing or arranging at least one tracer source in at least one zone or section of the well, the at least one tracer source having a release rate to a well fluid as a function of the at least one characteristic and/or condition of the well;
measuring tracer released from the at least one tracer source in well fluid;
based on the measured tracer data determining at least one well characteristic and/or condition of at least one zone or section of the hydrocarbon well.

The method may comprise estimating and/or predicting future and/or past measured tracer concentrations. The method may comprise extrapolating the calculated concentration as a function of time. The method may comprise determining and/or monitoring at least one characteristic and/or condition of the well at each location where the at least one tracer source is located or in the vicinity where the at least one tracer source is located. The method may comprise allocating a change in at least one characteristic and/or condition to a specific zone or location in the well. The method may comprise measuring at least one parameter of the well and/or at least one parameter of a zone or section of the well where the at least one tracer source is located. The method may comprise associating changes in the measured tracer data with changes in the well parameter data. The method may comprise associating changes in the well parameter data with changes in the measured tracer data. The method may comprise associating changes in the well parameter data with one or more deviations between the measured tracer data and the expected tracer data. The method may comprise providing at least one tracer source in at least one zone or section of a well by arranging, installing, positioning or locating at least one tracer source in at least one zone or section of a well.

Embodiments of the second aspect of the invention may include one or more features of the first aspect of the invention or its embodiments, or vice versa.

According to a third aspect of the invention there is provided a method of monitoring at least one characteristic and/or condition of a hydrocarbon well, the method comprising:
providing or arranging at least one tracer source in a known level of the well, the at least one tracer source having a release rate to a well fluid as a function of the at least one characteristic and/or condition of the well;
calculating expected tracer data from the at least one tracer source;
measuring tracer released from the at least one tracer source in well fluid; and
comparing the expected tracer data and measured tracer data to determine at least one well characteristic and/or condition of at least one zone or section of a hydrocarbon well.

The at least one zone or section may be at or in the vicinity of the one or more levels of the well where the at least one tracer source is located. The method may comprise preparing adjusted or calibrated expected tracer data based on the calculated and measured tracer data. The method may comprise monitoring at least one well characteristic and/or condition of the hydrocarbon well based on the adjusted or calibrated expected tracer data. The method may comprise providing or arranging at least one tracer source in a known level of a well, by installing, positioning and/or locating at least one tracer source in a known level of a well. The method may comprise providing or arranging each at least one tracer source in a different known level of a well. The method may comprise providing or tracer sources in known levels of a well. The method may comprise providing or arranging two or more tracer sources, each in a different known level of a well. The method may comprise estimating and/or predicting future and/or past expected tracer data. The method may comprise extrapolating the adjusted and/or calibrated expected tracer data as a function of time. The method may comprise comparing measured tracer data with adjusted and/or calibrated expected tracer data to monitor the at least one well characteristic and/or condition of the hydrocarbon well. The method may comprise creating a model. The model may be a machine learning model.

Embodiments of the third aspect of the invention may include one or more features of the first and/or second aspects of the invention or their embodiments, or vice versa.

According to a fourth aspect of the invention there is provided a method of monitoring at least one characteristic and/or condition of a hydrocarbon well, the method comprising:
providing or arranging at least one tracer source in a known level of the well, the at least one tracer source having a release rate to a well fluid as a function of the at least one characteristic and/or condition of the well;
calculating an expected tracer concentration of the at least one tracer in the well fluid;
measuring at least one concentration of the at least one tracer in the well fluid; and
comparing the calculated and measured concentration of the tracer to assess at least one well characteristic and/or condition of the hydrocarbon well.

The method may comprise allocating a change in at least one characteristic and/or condition to the well. The method may comprise allocating a change in at least one characteristic and/or condition to a specific zone or location in the well. The method may comprise measuring at least one well parameter and/or at least one well-related parameter. The method may comprise comparing the well parameter data with the at least one tracer concentration. The method may comprise comparing the well parameter data with the at least one tracer concentration as a function of time. The method may comprise associating changes in the well parameter data with changes at least one tracer concentration. The method may comprise associating changes in the tracer concentration with changes in the well parameter data. The method may comprise inferring that changes in one or more well parameter measurements correspond with one or more changes in tracer concentrations. The method may comprise inferring that changes in one or more tracer concentrations and one or more well parameter measurements corresponds with one or more changes in at least one characteristic and/or condition of a hydrocarbon well. The method may comprise providing or arranging at least one tracer source in a known level of a well, by installing, positioning and/or locating at least one tracer source in a known level of a well. The method may comprise providing or arranging each at least one tracer source in a different known level of a well. The method may comprise providing or tracer sources in known levels of a well. The method may comprise providing or arranging two or more tracer sources, each in a different known level of a well.

Embodiments of the fourth aspect of the invention may include one or more features of the first to third aspects of the invention or their embodiments, or vice versa.

According to a fifth aspect of the invention there is provided a method of determining a location of a change in at least one characteristic and/or condition of a hydrocarbon well, the method comprising:
providing or arranging two or more tracer source in known levels of the well, each tracer source configured to release tracer to a well fluid as a function of the at least one characteristic and/or condition of the well;
measuring at least one well parameter as a function of time;
measuring tracer data; and
based on the tracer data and the measured well parameter data inferring at least one location of a change in at least one characteristic and/or condition of a hydrocarbon well.

The method may comprise measuring tracer data for released from the two or more tracer source. Each tracer source may have a release rate to a well fluid as a function of the at least one characteristic and/or condition of the well. The method may comprise comparing the well parameter data with measured tracer data from each tracer source. The method may comprise comparing the well parameter data with measured tracer data from each tracer source as a function of time. The method may comprise associating changes in the well parameter data with changes in at least one tracer data measurements. The method may comprise associating changes in the at least one tracer data with changes in the well parameter data. The method may comprise inferring that changes in one or more well parameter measurements resulted in a change in tracer data measurements located at a known level in the well. The method may comprise comparing the timing of a change in at least one well parameter data with the measured tracer data of at least one tracer. The method may comprise comparing the timing of a change in measured tracer data of at least tracer with at least one well parameter data. The well parameter data may comprise at least one well parameter or at least one well-related well parameter. The at least one well parameter or at least one well-related well parameter may be selected from the group comprising pressure, temperature, ion data, flowrate, flowrate of a target and/or non-target fluid, viscosity, mass-density, solid components in the fluid, gas-rate, choke settings, water cut, gas to oil ratio; composition of a target or non-target fluid, well fluid type, injection rate, production rate, production rate of a nearby producing well, seismic activity in the vicinity of the well and/or reservoir, electromagnetic signal data and/or core sample data.

The measured tracer data may be selected from the group comprising tracer concentration, tracer mass, mass flow and/or tracer type.

Embodiments of the fifth aspect of the invention may include one or more features of the first to fourth aspects of the invention or their embodiments, or vice versa.

According to a sixth aspect of the invention there is provided a method of monitoring at least one characteristic and/or condition of a hydrocarbon well, the method comprising:
providing or arranging at least one tracer source in a known level of the well, the at least one tracer source having an expected release rate to a well fluid for a first data set of characteristics and/or conditions of a hydrocarbon well;
measuring at least one concentration of the at least one tracer in the well fluid;
comparing the expected tracer concentration with the at least one measured tracer concentrations; and
based on the at least one measured concentration identifying a second data set of characteristics and/or conditions of a hydrocarbon well.

The method may comprise providing or arranging at least one tracer source in a known level of a well, by installing, positioning and/or locating at least one tracer source in a known level of a well. The method may comprise providing or arranging each at least one tracer source in a different known level of a well. The method may comprise providing or tracer sources in known levels of a well. The method may comprise providing or arranging two or more tracer sources, each in a different known level of a well.

Embodiments of the sixth aspect of the invention may include one or more features of the first to fifth aspects of the invention or their embodiments, or vice versa.

According to a seventh aspect of the invention there is provided a method of assessing one or more conditions of a hydrocarbon well the method comprising:
providing or arranging at least one tracer source in a known level of the well, the at least one tracer source having at least a first expected release rate to a well fluid under at least a first well condition or characteristic and at least a second expected release rate to a well fluid under at least a second well condition or characteristic;
measuring at least one concentration of the at least one tracer; and
based on the at least one measured concentration monitoring the at least one condition or characteristic of a hydrocarbon well.

The method may comprise providing or arranging at least one tracer source in a known level of a well, by installing, positioning and/or locating at least one tracer source in a known level of a well. The method may comprise providing or arranging each at least one tracer source in a different known level of a well. The method may comprise providing or tracer sources in known levels of a well. The method may comprise providing or arranging two or more tracer sources, each in a different known level of a well.

Embodiments of the seventh aspect of the invention may include one or more features of the first to sixth aspects of the invention or their embodiments, or vice versa.

According to an eighth aspect of the invention there is provided a well monitoring system, comprising:
at least one sampling device for collecting well fluid samples at known sampling times;
a computing device configured to compare tracer data in one or more collected samples with an expected tracer data to determine at least one characteristic and/or condition of the well.

The well monitoring system may comprise at least one sensor configured to measure at least one well parameter. The at least one well parameter may be selected from the group comprising pressure, temperature, ion data, flowrate, flowrate of a target and/or non-target fluid, viscosity, mass-density, solid components in the fluid, gas-rate, choke settings, water cut, gas to oil ratio; a composition of a target or a non-target fluid and/or well fluid type injection rate, injection rate of a well with tracer system/source, injection rate of any related injector or producer hydrocarbon well, production rate of a nearby producing well, seismic activity in the vicinity of the well and/or reservoir, tracer data, electromagnetic signal data and core sample data. The computing device may comprise a database of well conditions or characteristics and associated tracer release profiles for each well condition or characteristic. The computing device may be configured to compare tracer data with a database of tracer release profiles for different well conditions and/or well characteristics and/or compare tracer data with well parameter data.

The information on the well condition and characteristic in each zone or over the entire well may be utilised to control the production flow and/or characterise the reservoir.

The system may comprise at least one tracer source. The at least one tracer source may be configured to be arranged, installed, positioned and/or located in a known level of a well. The system may comprise two or more tracer sources wherein each tracer source is configured to be arranged, installed, positioned and/or located in a different known level of a well.

Embodiments of the eighth aspect of the invention may include one or more features of the first to seventh aspects of the invention or their embodiments, or vice versa.

According to a ninth aspect of the invention there is provided a method of monitoring at least one characteristic and/or condition of a hydrocarbon well, wherein the well comprises at least one tracer source located at a known level of the well, the method comprising:
measuring at least one concentration of the at least one tracer in the well fluid; wherein the at least one tracer has a release rate to a well fluid as a function of the at least one characteristic and/or condition of the well; and
comparing the at least one tracer concentration with a database of the tracer release function signatures to determine at least one well characteristic and/or condition of the hydrocarbon well.

The method may comprise analysing the at least one measured tracer concentration by comparing the data set with a database of tracer release function profiles or signatures associated with each well condition and/or characteristic. The method may comprise analysing the tracer release data set to assess one or more properties or attributes of a physical condition of the well. The method may comprise measuring at least one well parameter. The method may comprise selecting one or more characteristics of the released tracer concentration based on the modelled well condition parameter. The method may comprise designing a tracer source having one or more release characteristics under different well conditions. The method may comprise simulating a tracer release response based on a well condition model to obtain a first simulated tracer release response data set. The method may comprise simulating a second tracer release response based on well condition model to obtain a second simulated tracer release response data set.

The method may comprise building a database of tracer release data, wherein the tracer release data is data collected from one or more tracer release tests performed on known well condition parameters. The method may comprise generating a library or database of tracer release data sets for each of the at least one tracer source corresponding to range of well characteristics and/or conditions. The method may comprise comparing the timing of a change in at least one well parameter data with the measured concentration of at least one tracer. The method may comprise comparing the timing of a change in measured concentration of at least tracer with at least one well parameter data. The method may comprise optimising the model by performing history matching. The method may comprise optimising the model by modifying the calculated tracer release function based on the observed release function. The modelling step may be repeated to generate a second simulated data set. This second simulated data set can be compared with the observed data and the first simulated data set. The method comprise optimising the model by adjusting and/comparing the first and second data set to determine which is better match to the observed tracer data.

The well may be one or more well in a well system. The well may be production well. The well may be a producer well. The production well may comprise at least one tracer source located at a known level in the production well. The well may be an injection or injector well. The injection or injector well may comprise at least one tracer source located at a known level in the injection or injector well. The well may be a producer well. The producer well may comprise at least one tracer source located at a known level in the producer well. The injection and/or producer well may comprise at least one tracer source located at a known level in the injection and/or producer well. The method may comprise providing or arranging at least one tracer source in a known level of a well, by installing, positioning and/or locating at least one tracer source in a known level of a well. The method may comprise providing or arranging each at least one tracer source in a different known level of a well. The method may comprise providing or tracer sources in known levels of a well. The method may comprise providing or arranging two or more tracer sources, each in a different known level of a well.

Embodiments of the ninth aspect of the invention may include one or more features of the first to eighth aspects of the invention or their embodiments, or vice versa.

According to a tenth aspect of the invention there is provided a method of monitoring at least one characteristic and/or condition related to a hydrocarbon well, wherein the well comprises at least one tracer source located at a known zone or section of the well, the method comprising:
providing measured tracer data from samples previously collected from the well fluid and based on the measured tracer data determining at least one characteristic and/or condition of at least one zone or section of the well;
wherein the at least one tracer source is configured to release tracer material to the well fluid as a function of the at least one characteristic and/or condition.

The method may comprise providing or arranging at least one tracer source in a known level of a well, by installing, positioning and/or locating at least one tracer source in a known level of a well. The method may comprise providing or arranging each at least one tracer source in a different known level of the well. The method may comprise providing or tracer sources in known levels of the well. The method may comprise providing or arranging two or more tracer sources, each in a different known level of the well.

Embodiments of the tenth aspect of the invention may include one or more features of the first to ninth aspects of the invention or their embodiments, or vice versa.

According to an eleventh aspect of the invention there is provided a well monitoring system, comprising:
one or more tracer source configured to release tracer material to the well fluid as a function of the at least one characteristic and/or condition of at least one zone or section of the well; and
a computing device configured to compare measure tracer data from the one or more tracer source with an expected tracer data to determine at least one characteristic and/or condition of the well.

The one or more tracer source may be configured to be arranged, installed, positioned and/or located in a known level of a well. The system may comprise two or more tracer sources wherein each tracer source is configured to be arranged, installed, positioned and/or located in a different known level of a well. The system may comprise at least one sensor configured to measure at least one well or well-related parameter.

The computing device may be configured to compare tracer data with a database of tracer release profiles for different well conditions and/or well characteristics. The computing device may be configured to compare tracer data with well parameter data or well-related parameter data. The computing device may be a processor. The system may comprise a sampling device for collecting well fluid samples at known sampling times.

Embodiments of the eleventh aspect of the invention may include one or more features of the first to tenth aspects of the invention or their embodiments, or vice versa.

According to a twelfth aspect of the invention there is provided a method of monitoring at least one characteristic and/or condition related to a hydrocarbon well, wherein a well comprises at least one tracer source located at a known zone or section in the well, the method comprising:
obtaining measured tracer data of tracer in a well fluid; wherein the at least one tracer source is configured to release tracer material to the well fluid as a function of the at least one characteristic and/or condition of at least one zone or section of the well;
obtaining or calculating expected tracer data; and
comparing the measured tracer data with the expected tracer data to determine at least one characteristic and/or condition of at least one zone or section of the well.

The method may comprise observing one or more deviations between the measured tracer data and the expected tracer data. The method may comprise measuring at least one parameter of the well, at least one parameter of an associated reservoir, at least one parameter of an associated well and/or at least one parameter of a zone or section of the well where the at least one tracer source is located. The method may comprise comparing the at least one parameter data with the measured tracer data and/or expected tracer data. The method may comprise associating changes in the well parameter data with changes in the measured tracer data. The method may comprise associating changes in the measured tracer data with changes in the well parameter data. The method may comprise associating changes in the well parameter data with one or more deviations between the measured tracer data and the expected tracer data. The method may comprise associating one or more deviations between the measured tracer data and the expected tracer data with changes in the well parameter data. The method may comprise inferring one or more deviations between the measured tracer data and the expected tracer data corresponds with changes in at least one characteristic and/or condition of at least one zone or section of the well. The at least one well parameter may be selected from the group comprising pressure, temperature, ion data, flowrate, flowrate of a target and/or non-target fluid, viscosity, mass-density, solid components in the fluid, gas-rate, choke settings, water cut, gas to oil ratio; composition of a target or non-target fluid, well fluid type, injection rate, production rate, production rate of a nearby producing well, seismic activity in the vicinity of the well and/or reservoir, electromagnetic signal data, tracer data, and/or core sample data. The measured tracer data and/or expected tracer data may be selected from the group comprising tracer concentration, tracer mass, mass flow and/or tracer type. The method may comprise obtaining measured tracer data by sampling well fluid and/or analysing the samples.

The at least one characteristic and/or conditions may be selected from the group comprising pressure, temperature, viscosity of a well fluid, a composition of a target or a non-target fluid, diffusion coefficient of the tracer, diffusion coefficient (D_m) of the tracer in a target fluid, tracer type; composition of a tracer matrix; composition of a tracer material; physical dimensions of the tracer system, molecular weight of tracer, water cut, well fluid type, and/or degree of exposure of tracer to target fluid. The method may comprise modelling tracer release from the at least one tracer source for at least one well characteristic and/or well condition of the well. The method may comprise generating a library or database of tracer release profiles for each well characteristic and/or well condition. The method may comprise comparing measured tracer data with the library or database of tracer release profiles to identify the at least one well characteristic and/or well condition of the well. Each tracer source may comprise at least one tracer material wherein the tracer material is selected from the group comprising chemical, fluorescent, phosphorescent, magnetic, DNA and radioactive compounds. The at least one tracer source may comprise a tracer material and a carrier. The tracer material may be configured to be released from the tracer carrier at a release rate dependent on at least one characteristic and/or condition of the well. The tracer material and/or the carrier may be well characteristic and/or well condition specific wherein tracer molecules may be released at different rates from the tracer source in response to a change of well characteristic and/or well condition in a section or zone of well in which the tracer source is located. The tracer material may be configured to be released from the tracer source at a first release rate under a first well condition and/or a first well characteristic in the section of the well the tracer source is located and the tracer material is configured to be released from the tracer source at a second release rate under a second well condition and/or second well characteristic in the section of the well the tracer source is located. The well may comprise two or more tracer sources, each tracer sources may be located in a known section or zone of the well and determining and/or monitoring at least one characteristic and/or condition of the well at at least one of the locations of tracer sources.

Embodiments of the twelfth aspect of the invention may include one or more features of the first to eleventh aspects of the invention or their embodiments, or vice versa.

According to a thirteenth aspect of the invention there is provided a method of monitoring at least one characteristic and/or condition related to a hydrocarbon well, wherein a well comprises at least one tracer source located at a known zone or section in the well, the method comprising:
collecting samples of well fluid;
analysing the samples to obtain measured tracer data; wherein the at least one tracer source is configured to release tracer material to a well fluid as a function of the at least one characteristic and/or condition of at least one zone or section of the well; obtaining or calculating expected tracer data; and
comparing the measured tracer data with the expected tracer data to determine at least one characteristic and/or condition of at least one zone or section of the well.

Embodiments of the thirteenth aspect of the invention may include one or more features of the first to twelfth aspects of the invention or their embodiments, or vice versa.

According to a fourteenth aspect of the invention there is provided a method of collecting samples for later analysis in monitoring at least one characteristic and/or condition related to a hydrocarbon well, wherein the well comprises at least one tracer source located at a known zone or section in the well, wherein the at least one tracer source is configured to release tracer material to the well fluid as a function of the at least one characteristic and/or condition of at least one zone or section of the well;
inducing production flow in the well; and
collecting samples downstream of the at least one tracer source at known sampling times.

Embodiments of the fourteenth aspect of the invention may include one or more features of the first to thirteenth aspects of the invention or their embodiments, or vice versa.

According to a fifteenth aspect of the invention there is provided a method of determining a location of a change in at least one characteristic and/or condition of a hydrocarbon well, wherein the well comprises two or more tracer sources in known levels of the well, each tracer source configured to release tracer to a well fluid as a function of the at least one characteristic and/or condition of the well; the method comprising:
measuring at least one well parameter as a function of time;
measuring tracer data; and
based on the measured tracer data and the measured well parameter data inferring at least one location of a change in at least one characteristic and/or condition of a hydrocarbon well.

The method may comprise determining at least one characteristic and/or condition of the hydrocarbon well. The method may comprise determining the position of at least one characteristic and/or condition of the hydrocarbon well. Each tracer source may have a release rate to a well fluid as a function of the at least one characteristic and/or condition of the well. The method may comprise measuring tracer data for tracer released from the two or more tracer sources into a well fluid.

Embodiments of the fifteenth aspect of the invention may include one or more features of the first to fourteenth aspects of the invention or their embodiments, or vice versa.

### BRIEF DESCRIPTION OF THE DRAWINGS

There will now be described, by way of example only, various embodiments of the invention with reference to the drawings, of which:

FIG. 1 is a schematic representation of a well with a tracer release system according to an embodiment of the invention;

FIGS. 2A to 2D are graphical representations of tracer release function curves for varying temperatures between 50° C. to 80° C.;

FIG. 3 is a graphical representation of a tracer release function parameter against temperature based on the curves of FIGS. 2A to 2D;

FIG. 4 is a graphical representation of an expected (predicted) tracer release function curve against time;

FIG. 5A is a graphical representation of a tracer release function curve based on calibrated observed tracer “A” concentration-based signal data points from a well. The uncalibrated tracer release function data is also shown (dotted line);

FIG. 5B is a graphical representation of a tracer release function curve based on calibrated observed tracer “B” concentration data from a well. The uncalibrated tracer release function data is also shown (dotted line);

FIG. 6A is a graphical representation of a calibrated tracer release function curve based on calibrated observed tracer “C” concentration data from a well. The uncalibrated tracer release function data is not shown;

FIG. 6B is a graphical representation of an expected (predicted) tracer release function curve for Tracer “C” based on extrapolated data from the calibrated observed tracer “C” concentration data in FIG. 6A;

FIG. 7 is a graphical representation of a power law functionality for three different predicted mass release as a function of time;

FIG. 8 is a graphical representation of a tracer release function curve based on calibrated observed tracer “C” concentration data from a well; expected tracer release function for tracer “C” over a period of 30 days (dotted line); and new observed data points over the predicted time period (triangular data points);

FIG. 9 is a graphical representation of a tracer release function curve based on calibrated observed tracer “E” concentration data from a well; expected tracer release function for tracer “E” over a period of 30 days (dotted line); and new observed data points over the predicted time period (triangular data points);

FIG. 10 is a graphical representation of a tracer release function curve based on calibrated observed tracer “F” concentration data from a well; expected tracer release function for tracer “F” over a period of 30 days (dotted line) and new observed data points over the predicted time period (triangular data points) on a logarithmic y-axis; The difference between calibrated function and trendline following new observed data points is displayed as a shaded area;

FIG. 11 is a graphical representation of a selection of well condition parameter data including water cut and well temperature against time;

FIG. 12A is a graphical representation of a tracer release function curve based on calibrated observed tracer “C” concentration data from a well; expected tracer release function for tracer “C” over a period of 30 days (dotted line); and new observed data points over the predicted time period (triangular data points) where the new observed data points are less than the expected tracer release function;

FIG. 12B is a graphical representation of a selection of well condition parameter data including water cut, well temperature and gas to oil ratio against time over the same period as the data in FIG. 12A was taken where the gas to oil ratio is un-measured;

FIG. 13 is a flow diagram representing steps of a method of determining well conditions and/or characteristics according to an embodiment of the invention;

FIG. 14 is a flow diagram representing steps of a method of determining well conditions and/or characteristics using a model according to another embodiment of the invention; and

FIG. 15 is a graphical representation of well parameter data (pressure, temperature and flow rate) of a well over time and measured tracer data.

### DETAILED DESCRIPTION OF PREFERRED EMBODIMENTS

There will now be described, by way of example only, various embodiments of the invention with reference to the drawings, of which:

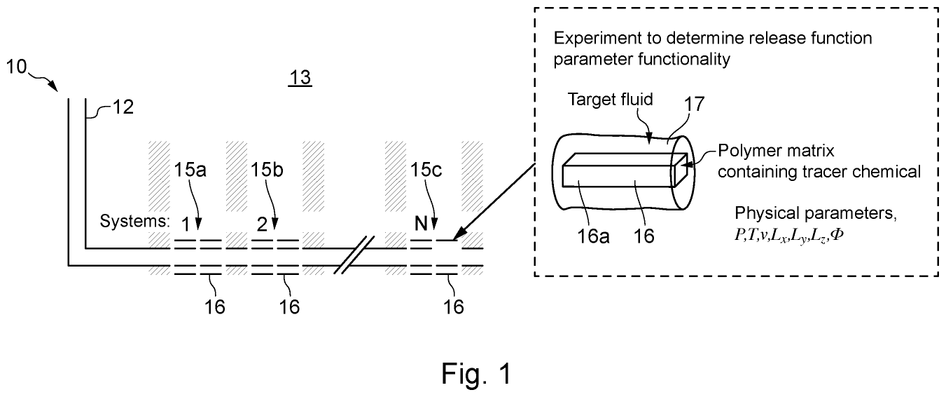

FIG. 1 is a simplified section through a production well 10. A central production tubing 12 is arranged in the well surrounded by annulus. Influx volumes of fluids enter the well from a reservoir 13 into the central production tubing 12 via separate influx locations. In this example, there are three influx locations 15a, 15b and 15c and three tracer release apparatus located in the proximity or near the influx locations. The dotted box in FIG. 1 shows an enlarged tracer source 16. Each tracer source 16 has a different tracer material 16a distinct for each influx location. In this example the tracers are designed to be released from the tracer source 16 when they are contacted by a target well fluid 17. Tracer measurements may be taken downstream of the tracer source and/or at surface to provide information on influx locations are producing and the rates of influx. The measurements may be taken by sampling well fluids or by real time measurements. The tracer systems are devised in a manner where the tracer is released in a predictable manner. The mass released as function of time can be described as a scalar function of time:

$$m = f ( t , α ) \qquad ( 1 )$$

where α is a specific set of parameters describing the physical condition at a given time.

In this example the parameter α is temperature. However, the parameter may alternatively or additionally be selected from the group comprising pressure (P), temperature (T), viscosity (v) of the target fluid of the system, specific composition of the target or non-target fluid (F), diffusion coefficient of the tracer chemical in the tracer system matrix, diffusion coefficient (D_m) of the tracer chemical in the target fluid, the tracer system matrix composition (C) physical dimensions of the tracer system (L_x, L_y, L_z), tracer molecular weight, oil API, and the extent at which the system is covered by the target fluid (φ).

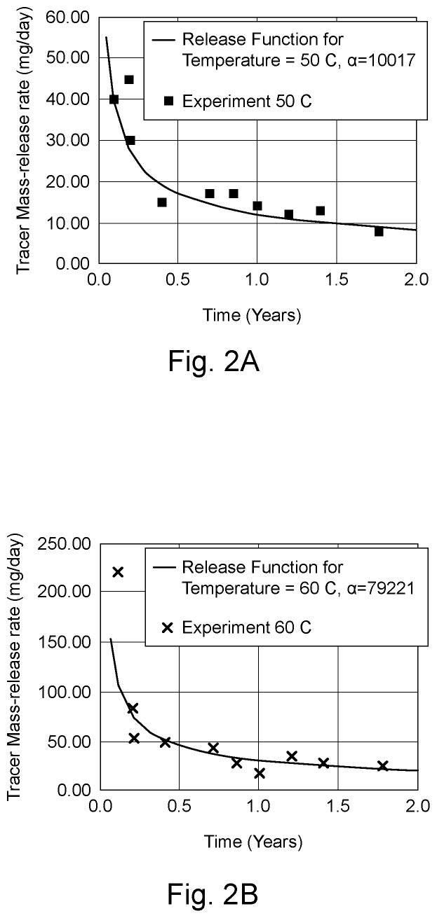

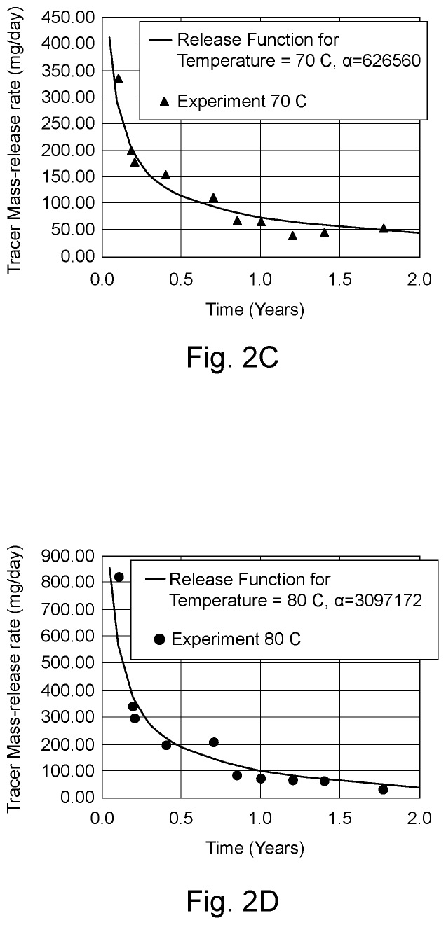

A library or database of the function dependency of the parameter in a may be collated by performing experiments where one parameter is changed while all other parameters are kept constant. FIGS. 2A to 2D show four experiment tracer release curves where the temperature is varied (50° C., 60° C., 70° C. and 80° C. in FIGS. 2A to 2D respectively) to find the temperature dependence of parameter α.

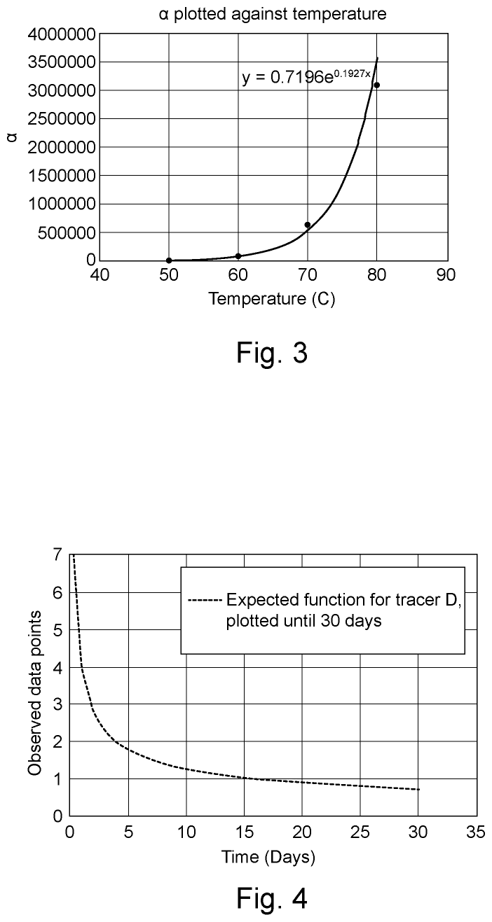

FIG. 3 correlates the temperature-dependence of parameter α, based on experiments of FIGS. 2A to 2D, to a mathematic expression which shows a good mathematical match to an exponential function, hence the mathematic expression becomes the temperature-dependent a. In a similar fashion, any other physical parameter-dependency of α may be determined. Some parameters are marginally subject to changes once installed in a well. For example physical dimensions of the tracer system (i.e. the size of the tracer source) typically stay constant over time. Some parameters depend on conditions that can be quasi-constant over extended periods of time or change gradually over time in the wells such as fluid viscosity (v), pressure (P) and temperature (T). Finally, some parameters such as the fraction at which the tracer systems are covered by the target fluid (φ) can change abruptly during the lifetime of the systems when placed in the wells, e.g., if a significant inflow of the system target phase is experienced.

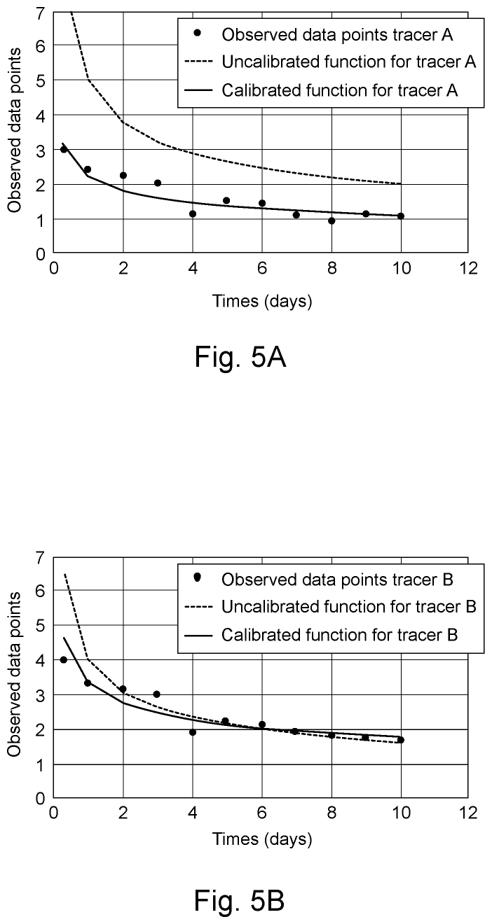

FIG. 4 shows an expected future release function generated for 30 days for a tracer in this example, tracer “D”. FIGS. 5A and 5B show calibration of tracer release function to observed tracer release data for tracer “A” and tracer “B” respectively, from the well. Calibration may allow accurate prediction of tracer release. Calibration is applied to observed tracer chemical data points from the well, from any time. In FIGS. 5A and 5B the time chosen starts from close to 0. In this example the calibration step assumes that the physical parameter-dependency are maintained. The release function for the well may be inaccurate for a number of reasons. A first possible scenario may be that one of the physical parameter dependencies of the release function may be inaccurate. A second scenario may be that any observed physical parameters from the well is inaccurate. A third scenario may be that an observed physical parameter from the well is unknown (if there is no downhole temperature gauge for example, the temperature would be unknown). In the case of the third scenario an initial expression can be impossible due to the lack of an input parameter (temperature), however an expression may still be based on observed tracer data point and other, known, physical parameters. A fourth possible scenario may be there are no observed physical parameters from the well. However in this scenario an expression may be based on observed tracer data points only.

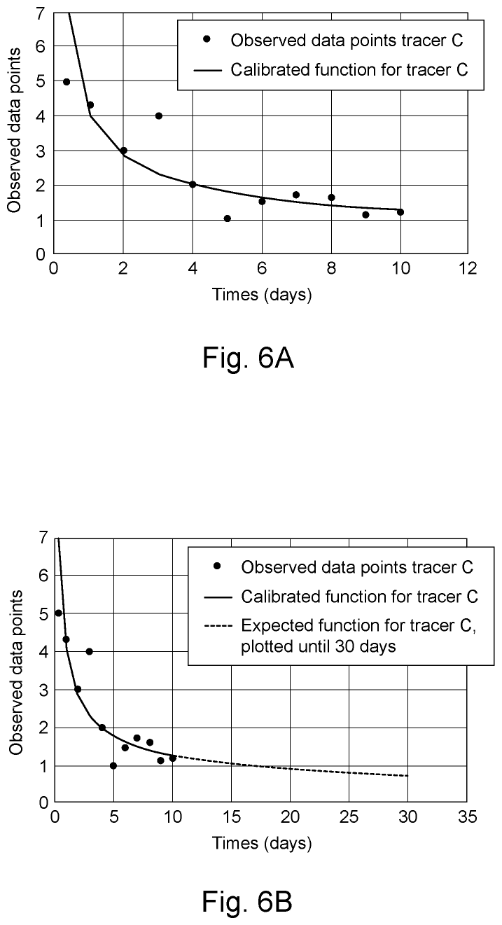

FIG. 6A shows a calibrated release function for tracer C from the well. The calibrated release function is based on uncalibrated function for tracer C (not shown). corrected using observed data points for tracer C. FIG. 6B shows an expected future release generated for day 10 onward building on the curve of FIG. 6A. The release function is extrapolated into the future, as the function is time dependent.

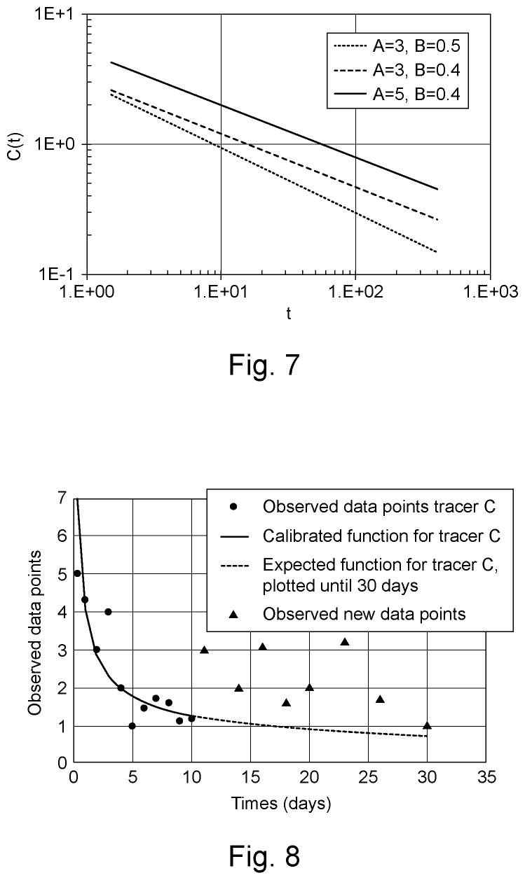

FIG. 7 shows power law functionality for the predicted mass release as a function of time. After establishing a release prediction, the prediction can be compared to observed well or tracer condition data points (other than those used in the calibration).

One example of a calibrated release function can be:

$$m = f ( t , α ) = A ( α ) \cdot 10 \hat{} ( - B ( α ) \cdot t ) \qquad ( 2 )$$

where A(α) and B(α) are constants for a given parameter value set, and that describe the power-law. As shown in FIG. 7, power-law functions are straight lines when plotted in log-log scale and the values of the constants A and B that define the functionality can be readily estimated from the linearity on log-log scale. Similar values of the constant B for two functions yield parallel curves (full line and dashed line in FIG. 7). Different values of B for two functions yield different inclination for the corresponding linear curves (e.g., dashed line and dotted line in FIG. 7). The values of A(α) and B(α) are constants for a given physical parameter set but take different values for another given parameter set. The predicted behaviour may be any function developed in any manner, similar to the initial model. The data points used for calibration can be any, not limited to the first data points, and may be only one data point. The predicted release-function depends on a number of physical parameters. If the values of one or more of the physical parameters such as temperature, pressure etc, changes, a difference between expected tracer release function and observed tracer release data may occur.

FIG. 8 shows a tracer release for Tracer C. The curve shows a calibrated data (full line) based on observed tracer C data. The curve also shows an expected release data (dashed line) from day 10 until day 30. FIG. 8 shows there is a difference between the expected release function (dashed line) and the observed tracer data points (black triangles). The observed tracer data points show a higher tracer release than expected from day 10 indicating that one of the physical parameters may have changed (between Day 10 and Day 11 in a way that, in this example, produces higher tracer release. A change in mass release function due to changes in physical parameters can be exploited to infer a value of a physical parameter by comparison of function after a change occurs to previous function. Specifically, the observed behaviour up to a time when an abrupt change is seen (Day 10/11) can be matched and compared to behaviour known from experiments. In most cases, several tracers will be installed for a given well—with unique tracer chemicals for each location. By comparing observed behaviour to experimentally determined release function, physical conditions in all locations where a system is installed can be determined.

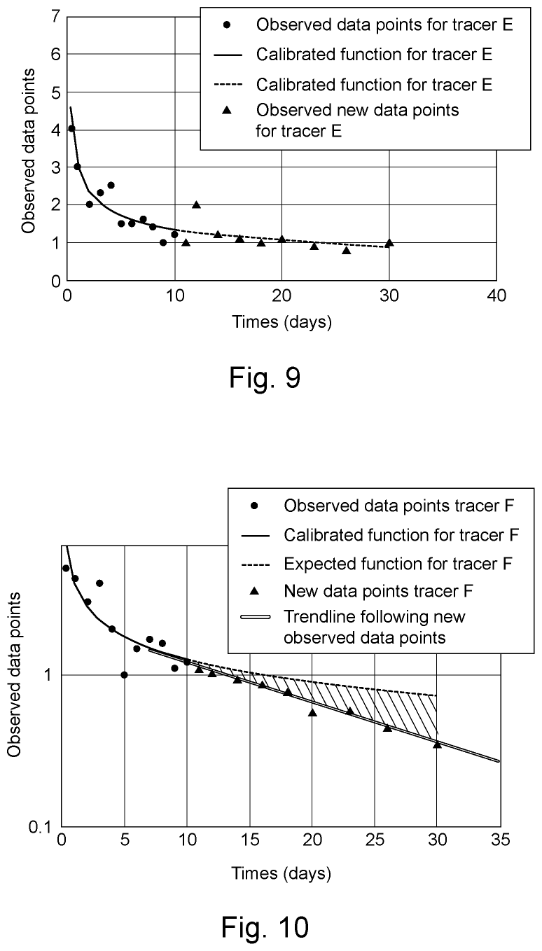

FIG. 9 shows a tracer release curve for Tracer E which is released in the same well as Tracer C. The observed release data points for Tracer E do not deviate from its expected release function indicating that the change in physical condition which affect Tracer C do not affect Tracer E.

FIG. 10 is a graph of a calibrated data response over time based on observed data points. The graph has an expected future tracer response shown as a dotted line. The graph also shows a trendline shown as following new observed data points for tracer F on a logarithmic y-axis. The graph illustrates the difference between expected tracer release function and new actual tracer release trendline with shading. As shown in FIG. 10, the difference between the expected tracer release function and the new data points is initially small and gradually increasing over time. In this case, the new actual trendline function is applied to help observe the gradually increasing difference, and further visual support can be provided for example by using the area shading. A comparison of expected and observed tracer release function may be done by eye (such as in this example), by machine analysis, or a mathematical and/or statistical analysis. The comparison may be sophisticated and detailed, such that each data point individually can be evaluated whether they are deviating, or it can be for many data points grouped. The difference between the function and the observed data points can be of any magnitude.

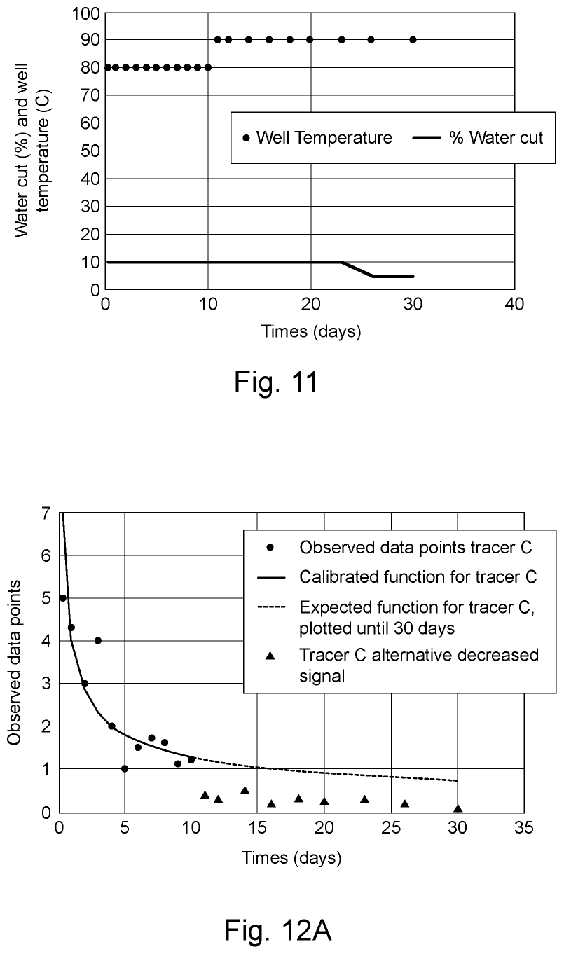

FIG. 11 shows deviations of physical well parameters over time. FIG. 11 shows two physical parameters, temperature (dots) and water cut (line) over a 30 day period. The data shows two significant changes, temperature increases from 80° C. until 90° C. on day 10, and water cut decreases from day 23 to day 25.

In general, a change in well physical parameter may be instantaneous, the full change happening between two measured times such as the example, or it can be a gradual change, happening over a long time period. It could be that the parameter later changes to another value than the first change. An important requirement is that a change in a parameter is observed relative to the time. Examples of physical parameters can be pressure, temperature, ion data, flowrate for target and non-target fluids, viscosity, mass-density, solid components in the fluid, gas-rate, GOR, water cut, choke settings, injection rate of the well with tracer system/source, injection rate of any related injector or producer hydrocarbon well, production rate of a nearby producing well, observed seismic activity in the vicinity of the well and/or reservoir data such as seismic data, electromagnetic signal data, tracer data, and/or data extracted from a core sample. The parameters can be measured for the well with the tracers at any location of the well or flow system. Parameters can be measured at nearby injector well or producer wells that could possibly affect the tracer release-rate. Alternative metering systems such as seismic, CPI, production logging, etc may be used. The observed physical parameters do not need to be part of an initial model or a prediction model. The system may be used to associate changes in tracer to changes in well data. Using the example where Tracer C and Tracer E are two tracers installed in a well, similar to the example described in FIG. 1. Physical well parameters may be observed such as temperature and water as illustrated in FIG. 11.

By correlating a time point of a tracer deviation with a time point of a physical well parameter deviation, the location in the well, where the physical parameter change occurs may be determined. For example by associating a deviation in release of tracer C with a change in a physical well parameter, the location source of tracer C determines where the change in physical parameter has occurred. In this example the overall temperature increase in an oil well could be due to nearby injection of hot gas used for enhanced production purposes, and this hot gas can inflow at one of the tracer locations. But the observed physical parameter (well temperature) does identify where, because it is a measurement of the well in general, after all produced fluids are mixed together.

By comparing the tracer release curves in FIGS. 8 and 9 and the well parameter data of FIG. 11 and correlating timing between tracer release changes and well parameter changes it may be concluded that tracer C concentrations in the well fluids shown in FIG. 8 increases when the well temperature increases on Day 10, hence the location of Tracer C is likely where very hot fluids enter the wellbore and increases the overall well temperature. It may also be concluded that Tracer E release does not increase when temperature increases on Day 10, hence the location of Tracer E is not where the hot fluids enters the wellbore. It may also be concluded that the water cut decreases from 10% to 5% at day 23, but the timing of the current analysis with selected calibration data points does not correlate to a change in either Tracer C or Tracer E. From this it may be inferred that Tracer C and Tracer E are not located in sections of the well where water cut occurs. Alternatively, it may be inferred that the water cut change is not significant enough to make an observable difference in the tracer signals of Tracer C or Tracer E.

The above comparison analysis relates to an overlap of timing. However, synchronisation between well physical parameter change and a change in tracer data is not necessary. A gradual change in a well physical parameter could lead to a sudden change in tracer release signal. There could be a difference between when the deviation occurs for the well physical parameter and when the deviation happens or start happening for the tracer release. The time when the comparison was made, or when a calibration was made can be any period: During a clean-up period of the well, during a re-start, or during normal production of the well. The observed data may be compared with model data to confirm that it matches with the model physics. In the event of an increase in well temperature its effect on temperature may be modelled and the observed data monitored to confirm that the correlation is correct. However scenarios may arise where the observed data is different to the expected data. In such cases, an unmeasured physical parameter may be responsible for the offset in the expected data. As an example, an unmeasured physical parameter may decrease a to a larger degree than the observed physical parameter has increased α.

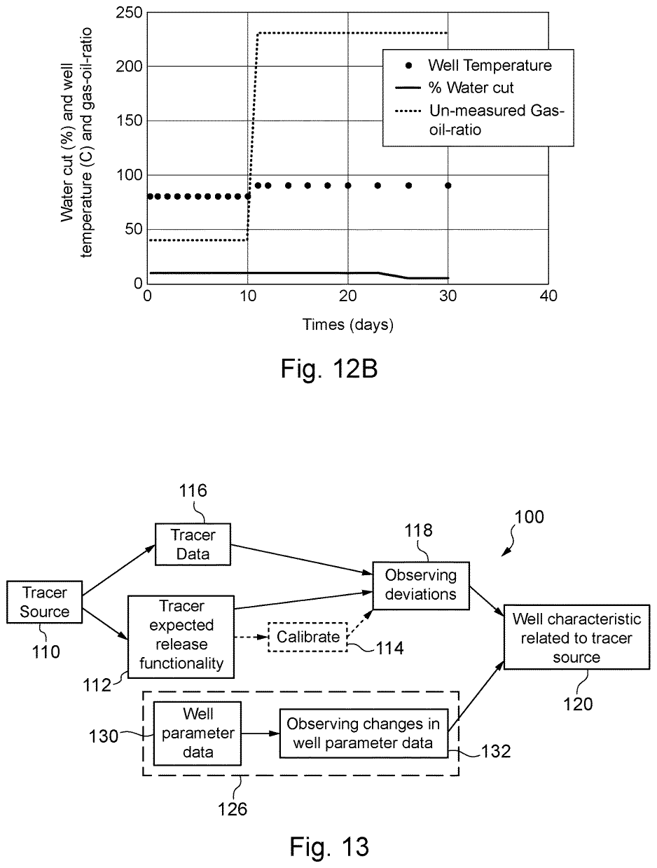

FIG. 12A shows an observed and expected tracer release curve for tracer “C” over a 30 day period. FIG. 12B shows observed well parameter measurements over the same period as FIG. 12A. The observed tracer data (black triangular points) for day 11 to day 30 is lower than the expected tracer release shown as the dotted line in FIG. 12A. FIG. 12B shows the well temperature increased from 80 degrees to 90 degrees on day 10. Using the known properties of Tracer C such an increase in well temperature would result in an increase in tracer release. However as shown in FIG. 12A instead of an expected tracer increase, a decrease in tracer release is observed (black triangles). Using the understanding of the well mentioned earlier, a likely explanation is a higher gas-oil ratio is present in the well. Tracer C is an oil tracer and an increase in gas-oil ratio would result in a reduction of exposure of the tracer system to oil in that location which in turn would reduce the release of tracer. This would account for the decrease in the observed tracer concentrations at day 11. This decrease offsets the expected tracer increase due to increased temperature of hot gas. The net result is an overall reduction in Tracer C seen in the observed data. In this case the parameter set has two changing physical parameters: i) temperature and ii) exposure to target fluid. Temperature may change at a specific time.

FIG. 13 provides a summary of an example process 100 to determine changes in physical condition and/or characteristics in section of a well downhole. The well has a tracer source 110 deployed in a well as discussed above in relation to FIG. 1. Each tracer in the tracer system has a defined, expected release function 112 under known conditions. The expected release function may be dependent on physical parameters. The expected release function may be developed by experimental data as described previously in relation to FIGS. 2A to 12B. Additionally or alternatively field data may be used to produce an expected release function based on previous experiment data and/or using a database of previously obtained concentrations and well physical parameters from one or many wells. After establishing the expected release function, the expected release function may be optionally calibrated 114 (Dotted box) as described previously in relation to FIGS. 4 to 12B. Tracer data 116 taken from released tracer in the well is compared to the expected release function. If there is any deviation 118 between the expected and observed release it is tracked and the timing noted. Using knowledge of the tracer type and the tracer release profile a well characteristic or condition 120 in the section of the well where the tracer source is located may be determined. As shown in the dashed box 126 of FIG. 13, optionally well parameters 130 may be measured over time. Any observed changes 132 in the well parameters are noted and tracked. The observed timing of the tracer deviation may be correlated with the observed timing of change in well parameter data. If there is a match in timing, the release function for the tracer may be monitored to confirm that the change in release function is in accordance with a change in the physical parameter observed. Conclusions may be drawn about the physical parameter changed for the location in the well of that tracer source 130.

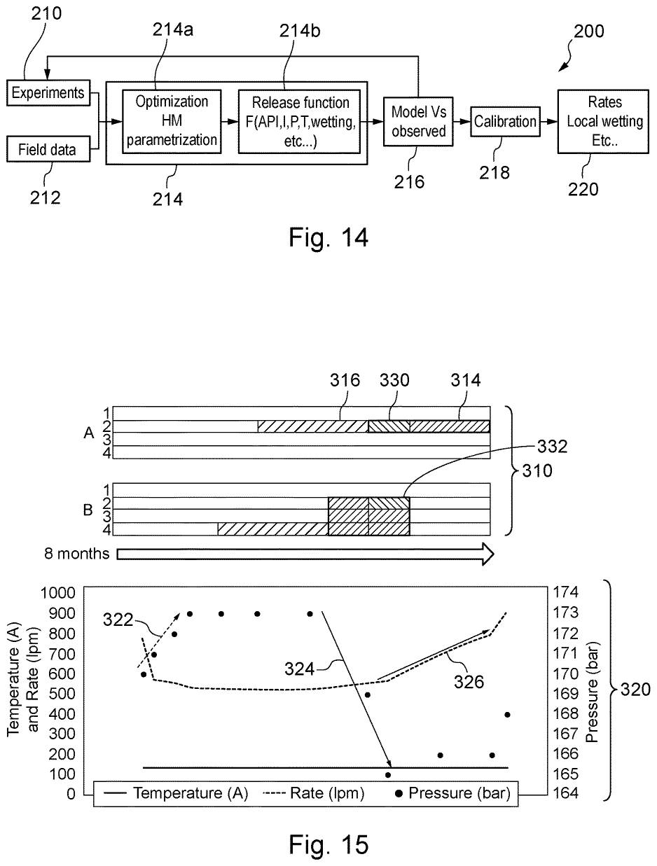

FIG. 14 provides a summary of another example process 200 which used a tracer release function model 214. Similarly, the well has a tracer system deployed in a well and experimental data (step 210) and field data (Step 212) are collected as discussed in FIG. 13. Field data comprising tracer concentrations from samples of well fluid are measured. The field data may optionally comprise well parameter measurements. The experimental data showing the relationship between parameter value and tracer release function may be stored to generate a library of release function profiles associated with the condition or characteristics of the well (Step 214b). In this example the relationship between API, pressure, temperature and wetting etc and tracer release is stored. From this knowledge of known and expected tracer release function under a variety of well conditions and characteristics are collated in this example in the model (Step 214). This knowledge will iteratively improve over time using experimental data and operation data through machine learning. Once the known tracer responses have been logged, anomalies may be readily recognisable. With additional data gathered against the well type and/or well conditions or characteristics, these anomalies will become associated with known well conditions or known well geometries. A database of known anomalies can then be collated using for example machine learning so that anomalies, which may include unexpected deviation of tracer release function under certain conditions or characteristics may be detected. Optionally the data may be calibrated (Step 218) using observed tracer data points for other tracers to reach a prediction of tracer release function over time. The database of release function profiles can be referenced during real measurement operations in order to characterise conditions or characteristics of the well (Step 220). In this example information on rates and local wetting may be determined. Such analysis can be performed using software algorithms, and the tracer release profiles or signatures may be stored as data sets within the database. The tracer release function profiles may be compared with the observed data to determine conditions or characteristics of the well. This may be by recognition of, for example, determining a change in well parameter or may be by inferring a change in an unknown unmeasured parameter by comparison with the data. The methodology to extract the information can be manual or be automated by using computer code and programs. In practice this will significantly ease the work involved in extracting the information. Once available in electronic form the data can also be conveniently illustrated together with other data.

In an example data set two physical parameters: temperature and exposure to target fluid may be considered. If the data shows that the there is a change in temperature but the exposure to target fluid remains the constant, using history-matching this physical parameter-scenario with observed tracer mass-release the following conclusions can be determined. As the exposure to target fluid does not change, any changes in value of A(α) and B(α) is not because of exposure to target fluid. The temperature parameter changes at the same time as the value of A(α), it is likely that the change in temperature is the reason for the change in the value of A(α). The temperature parameter changes at the same time as the value of B(α) starts to change trend, it is also probable that the temperature change is the reason for the change in the B(α) value. A model of the tracer system in the well may be generated, and may include library of release function profiles for various well conditions and characteristics based on the according to the modelled conditions experimental data. The measured tracer concentration may be run on the model which compares the modelled tracer release with observed tracer release to determine a well condition and/or characteristic. The model 214 can then be optimised by history matching (step 214a) and modifying the calculated tracer release function. In this example after a first simulated data set is generated, the model 214 may be optimised by history matching and the data simulation may be repeated to generate a second simulated data set. This data set can be compared with the observed data and the first simulated data set. It can be determined whether the second data set is better match to the observed tracer data.

As an alternative to the above, or as a subsequent method, the model may be trained or optimised using tracer release data from a well having two or more known conditions or characteristics. The model may be optimised by collating data set from multiple known well conditions or characteristics. Population of the model with tracer release function profiles from multiple combinations of well conditions or characteristics may be used to further optimise the model. The methodology to extract the relationship between condition/characteristic of a zone or section of a well and tracer data can be performed manually or automatically by using computer coding and programs. Once available in electronic form the tracer data and well parameter data may also be conveniently illustrated together with other data. One such example is provided in FIG. 15.

FIG. 15 provides a graphical representation of well data and tracer data from two tracer types, tracer type “A” being a water tracer, sensitive to the presence of water. Tracer type “B” being an oil tracer, sensitive to the presence of oil. Four different water tracer sources (A1 to A4) were placed at known locations in a well. Four different oil tracer sources (B1 to B4) were also placed at known locations in the well. In this example they are placed in a production well. It will be appreciated that in other examples they could be placed in an injector well, producer well, or other wells associated with a well system. FIG. 15 provides a graphical interpretation of when and where significant changes happen in a well. The chart 310 tracks tracer concentration data over a period of time, in this example 8 months. The strength of the shading represents the amplitude of the tracer signal detected. The dark shading shown as 314 represents a high tracer concentration in comparison to the light shading shown for example as 316 in FIG. 15 representing lower tracer concentration. The chart also shows “boxed” sections 330, 332 which represent an interpretation of significant tracer changes which should be compared to production (well parameter) data. The “boxed” sections may be automatically flagged as significant changes in tracer and/or well parameter activity. It may be performed by computer software, machine learning, artificial intelligence and/or by human analyst. The graph section 320 shows well parameter data for the overlapping time period (8 months). In this example, the parameters temperature and flow rate are scaled to the left y-axis, and the parameter pressure scaled to the right y-axis. Arrows 322, 324 and 326 drawn in the FIG. 15 highlight significant changes in a particular well parameter which, in the graphical representation, is interpreted as significant well changes to compare to changes in tracer data of chart 310. The graphical representation supports correlation between tracer source locations and production data, the following is an example interpretation based on this example graphic combined with generic tracer and production data understanding. In this example data set shown in FIG. 15, temperature shown as the full line is shown to remain constant. It can be inferred that changes in tracer release shown in chart 310 is not related to a change in temperature. The pressure data initially increases from 170 bar to 173 bar as highlighted by arrow 322, the pressure was constant at around 173 bar for a number of months. Over this constant-pressure time period a slight increase in tracer release was observed for tracer A2 and B4.

Although these parameter changes were observed, in this example they were not boxed, meaning that the graphical representation did not assign them a significant meaning. The graphical representation may be programmed based on selected criteria to determine whether changes in tracer and/or well parameter data are significant. The programming and selection criteria may differ from various analytical techniques. The programming and selection criteria may differ depending on the type of well, type of tracer and/or type of well parameters being monitored or determined. For this example, the graphical representation is trusted, and any changes which are not considered significant are ignored.

The pressure decreased significantly from approximately 173 bar to 166 bar as highlighted by arrow 324. Over this time period a significant increase in tracer release was observed for tracers A2, B2, B3 and B4. It can be inferred that these sections of the well are affected by an event causing the pressure to decrease. The tracer release increase may possibly not be caused not by pressure directly, but an un-measured parameter that would both cause tracer release to increase and the pressure to decrease simultaneously. If the pressure decrease directly caused the increase in tracer release, one could expect the tracer increase to be maintained for as long as the pressure was at 166 bar, assuming that is how these tracer sources react to pressure. This assumption may be based on earlier lab tracer-source release experiments where tracer types B2, B3, and B4, are exposed to decreasing pressure parameters while other parameters are kept constant, and observing that the there was no increase in tracer release under decreasing pressure conditions. By looking into the details of the interpreted significant changes (data that is boxed), it seems that the change in release of tracer from source A2 mostly happened after the change in pressure. While this doesn't disqualify the correlation between pressure and A2, there is another well parameter that appears to overlap much better with A2. Arrow 326 represents a gradual increase in flow rate over the last few months (approximately month 5.5 to 8). This flow rate increase fully overlaps with the A2 interpreted increase (boxed area 330 of A2), and furthermore, the pressure, or pressure-related effect causing the “B” (oil) tracers (B2 to B4) to increase, seems to stop, while tracer A2 (water tracer) keeps increasing. Furthermore, because A2 is a different type of tracer source (water sensitive), A2 it may be insensitive to the potentially unmeasured production event which had affected B2, B3, and B4 (at month 4.5 to 6).

To summarise, the graphical presentation shown in FIG. 15 provides tracer data from tracer sources and production (well parameter) data. The graphical representation provides an interpretation on when sufficiently significant changes happen to the production data and tracer data. In this example, an analyst may review the graphical presentation and infer correlations between the two sets of data to allocate production data to the specific locations of the tracer sources in a well. The graphical presentation may show expected behaviour of tracer release to specific production data. The graphical presentation may also show the effects of any unmeasured well parameter effects on tracer release in the various sections of the well.

The methods described above do not require one single set of terms, as one can do mathematical transformations of terms and use the same technique. It will be appreciated that the use of time of as term, function or unit of quantification may be replaced by a proxy of time such as cumulative produced volume of oil, water and/or gas. Additionally, or alternatively a combination of the date and time may be used. It will also be appreciated that alternative terms, units, and functions for the tracer data may be used including concentration (mass per mass or mass per volume or volume per volume or volume per mass), mass flow (gram per hour), mass (kilo).

The method, system and/or parts of the method and system may be implemented using a computer program. The method, system and/or parts of the method and system may be implemented using machine learning. Data, data points, algorithms and functions for the method and system can be used and/or stored in a database and/or a removable storage device.

The invention may provide a system and methods of monitoring at least one characteristic and/or condition related to a hydrocarbon well wherein a well comprises at least one tracer source located at a known zone or section in the well. The method may comprise measuring tracer released from the at least one tracer source into well fluid; wherein the at least one tracer source may be configured to release tracer material to the well fluid as a function of the at least one characteristic and/or condition of at least one zone or section of the well.

The method according to an embodiment of the invention may be able to identify one or more zones or locations of a well where well characteristics and/or well conditions are changing based on the release response from tracers in the specific zone or section of the well. By analysing the tracer data and pattern of tracers it may be possible to determine qualitatively and/or quantitatively the changes in well characteristics and/or well conditions. The method according to an embodiment of the invention may allow the well characteristics and/or well conditions to be determined by analysis of the tracer release data from the tracers source located in the well compared with a database of tracer response signatures for a wide range of well characteristics and/or well conditions.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers. Furthermore, relative terms such as”, “downstream”, “upstream” and the like are used herein to indicate directions and locations as they apply to the appended drawings and will not be construed as limiting the invention and features thereof to particular arrangements or orientations. Likewise, the term “outlet” shall be construed as being an opening which, dependent on the direction of the movement of a fluid and may also serve as an “inlet”, and vice versa.

The foregoing description of the invention has been presented for the purposes of illustration and description and is not intended to be exhaustive or to limit the invention to the precise form disclosed. The described embodiments were chosen and described in order to best explain the principles of the invention and its practical application to thereby enable others skilled in the art to best utilise the invention in various embodiments and with various modifications as are suited to the particular use contemplated. Therefore, further modifications or improvements may be incorporated without departing from the scope of the invention as defined by the appended claims.

Various modifications to the above-described embodiments may be made within the scope of the invention, and the invention extends to combinations of features other than those expressly claimed herein.

1. A method of monitoring at least one characteristic and/or condition related to a hydrocarbon well, wherein a well comprises at least one tracer source located at a known zone or section in the well, the method comprising:
obtaining measured tracer data of tracer in a well fluid, wherein the at least one tracer source is configured to release tracer material to the well fluid as a function of the at least one characteristic and/or condition of at least one zone or section of the well;
obtaining or calculating expected tracer data; and
comparing the measured tracer data with the expected tracer data to determine at least one characteristic and/or condition of at least one zone or section of the well.

2. The method according to claim 1 comprising observing one or more deviations between the measured tracer data and the expected tracer data.

3. The method according to claim 1 comprising measuring at least one parameter of the well, at least one parameter of an associated reservoir, at least one parameter of an associated well and/or at least one parameter of a zone or section of the well where the at least one tracer source is located.

4. The method according to claim 3 comprising comparing the at least one parameter data with the measured tracer data and/or expected tracer data.

5. The method according to claim 3 comprising associating changes in the well parameter data with changes in the measured tracer data.

6. The method according to claim 3 comprising associating changes in the well parameter data with one or more deviations between the measured tracer data and the expected tracer data.

7. The method according to claim 3 comprising inferring one or more deviations between the measured tracer data and the expected tracer data corresponds with changes in at least one characteristic and/or condition of at least one zone or section of the well.

8. The method according to claim 3 wherein the at least one well parameter is selected from the group comprising pressure, temperature, ion data, flowrate, flowrate of a target and/or non-target fluid, viscosity, mass-density, solid components in the fluid, gas-rate, choke settings, water cut, gas to oil ratio; composition of a target or non-target fluid, well fluid type, injection rate, production rate, production rate of a nearby producing well, seismic activity in the vicinity of the well and/or reservoir, electromagnetic signal data, tracer data, and/or core sample data.

9. The method according to claim 1 wherein the measured tracer data and/or expected tracer data is selected from the group comprising tracer concentration, tracer mass, mass flow and/or tracer type.

10. The method according to claim 1 comprising obtaining measured tracer data by sampling well fluid and analysing the samples.

11. The method according to claim 1 wherein the at least one characteristic and/or conditions is selected from the group comprising pressure, temperature, viscosity of a well fluid, a composition of a target or a non-target fluid, diffusion coefficient of the tracer, diffusion coefficient (D_m) of the tracer in a target fluid, tracer type; composition of a tracer matrix; composition of a tracer material; physical dimensions of the tracer system, molecular weight of tracer, water cut, well fluid type, and/or degree of exposure of tracer to target fluid.

12. The method according to claim 1 comprising modelling tracer release from the at least one tracer source for at least one well characteristic and/or well condition of the well.

13. The method according to claim 1 comprising generating a library or database of tracer release profiles for each well characteristic and/or well condition.

14. The method according to claim 13 comprising comparing measured tracer data with the library or database of tracer release profiles to identify the at least one well characteristic and/or well condition of the well.

15. The method according to claim 1 wherein each tracer source comprises at least one tracer material wherein the tracer material is selected from the group comprising chemical, fluorescent, phosphorescent, magnetic, DNA and radioactive compounds.

16. The method according to claim 1 wherein the at least one tracer source comprises a tracer material and a carrier.

17. The method according to claim 16 wherein the tracer material is configured to be released from the tracer carrier at a release rate dependent on at least one characteristic and/or condition of the well.

18. The method according to claim 16 wherein the tracer material and/or the carrier are well characteristic and/or well condition specific wherein tracer molecules will be released at different rates from the tracer source in response to a change of well characteristic and/or well condition in a section or zone of well in which the tracer source is located.

19. The method according to claim 16 wherein tracer material is configured to be released from the tracer source at a first release rate under a first well condition and/or first well characteristic in the section of the well the tracer source is located and the tracer material is configured to be released from the tracer source at a second release rate under a second well condition and/or second well characteristic in the section of the well the tracer source is located.

20. The method according to claim 1 wherein the well comprises two or more tracer sources, each tracer sources located in a known section or zone of the well and determining and/or monitoring at least one characteristic and/or condition of the well at at least one of the locations of tracer sources.

21. A well monitoring system, comprising:
one or more tracer sources configured to release tracer material to the well fluid as a function of the at least one characteristic and/or condition of at least one zone or section of the well; and
a computing device configured to compare measured tracer data from the one or more tracer sources with an expected tracer data to determine at least one characteristic and/or condition of the well.

22. The system according to claim 21 comprises at least one sensor configured to measure at least one well parameter or well-related parameter.

23. The system according to claim 21 wherein the computing device is configured to compare tracer data with a database of tracer release profiles for different well conditions and/or well characteristics and/or compare tracer data with well parameter data or well-related parameter data.

24. A method of monitoring at least one characteristic and/or condition related to a hydrocarbon well, wherein a well comprises at least one tracer source located at a known zone or section in the well, the method comprising:
collecting samples of well fluid;
analysing the samples to obtain measured tracer data, wherein the at least one tracer source is configured to release tracer material to the well fluid as a function of the at least one characteristic and/or condition of at least one zone or section of the well;
obtaining or calculating expected tracer data; and
comparing the measured tracer data with the expected tracer data to determine at least one characteristic and/or condition of at least one zone or section of the well.
